(19) United States
(12) Patent Application Publication
(10) Pub. No.: US 2024/0335162 A1
(43) Pub. Date: Oct. 10, 2024

(54) CUSTOMIZED ORAL CARE PRODUCTS

(71) Applicant: Adent ApS, Copenhagen K (DK)

(72) Inventors: Richard BUNDSGAARD, Copenhagen K (DK); Thøger BUNDSGAARD, Copenhagen K (DK)

(73) Assignee: Adent ApS, Copenhagen K (DK)

(21) Appl. No.: 18/707,034

(22) PCT Filed: Nov. 11, 2022
(86) PCT No.: PCT/EP2022/081564

§ 371 Date: May 2, 2024

(30) Foreign Application Priority Data
Nov. 15, 2021 (DK) PA202170559

(51) Int. Cl. A61B 5/00; A61Q 11/00; G16H 30/00

(52) U.S. Cl. CPC A61B 5/4547; A61Q 11/00; G16H 30/00

(57) ABSTRACT

A method for remotely providing customized oral care means to a person by obtaining, using the mobile device (40), at least one digital image (1) of the person's (30) oral cavity (31) and additional non-image data (2) comprising anamnestic information about the person (30). The digital image (1) is processed using a statistical algorithm (20) to extract at least one dental feature (3) present in the oral cavity (31), and an oral cavity profile (5) is assigned to the person (30) based on the identified dental features (3) and the non-image data (2). Recommended oral care means (6) are determined by statistical matching with the assigned oral cavity profile (5) and presented on the mobile device (40) or custom manufactured and provided for the person (30).

### TECHNICAL FIELD

The present disclosure relates generally to the field of oral health, and more particularly to a method for providing customized oral care means based on digital images and non-image data obtained using a mobile device with a camera.

### BACKGROUND

Oral health care based on an assessment of the state of the oral cavity and using the proper oral care means based on such an assessment has many advantages, most importantly for early intervention against and preventive treatment of oral health problems. However, many people do not receive proper oral health assessment and therefore have little or poor access proper oral care means, for several reasons. For example, they may live in rural areas far from a dental clinic and may, hence, not have access to oral health assessment, or they may not have the economic means to consult with a dental professional. Further, many people may be disinclined to spend time and money on an initial oral health assessment or on regular dental checkups if they are not experiencing any apparent oral health problem, despite the fact that people may unknowingly have symptoms of compromised oral health or unknowingly be at risk of developing an oral health problem. In other cases, people may be experiencing a certain oral health problem or symptoms of an oral disease but may decide to wait before consulting with a professional, hoping that the problem or symptoms will go away. All these example scenarios are problematic since early and proper oral health assessment allows for early intervention and preventive treatment, using for example customized or custom-selected oral care ingredients and products, thus preventing or reducing the occurrence or progress of many oral problems and diseases.

Methods and systems already exist for providing remote oral health assessment and additional guidance, such as dental treatment plans for remote patients using a mobile device with a camera, as also disclosed in the applicant's previous patent applications DK201970622/WO2021064112A1 (“Method for obtaining images for assessing oral health using a mobile device camera and a mirror”), DK201970623/WO2021064114A1 (“Method for assessing oral health using a mobile device”), DK202070136/WO2021175713A1 (“Dental feature identification for assessing oral health”), and DK202070180/WO2021191070A1 (“Oral health assessment for generating a dental treatment plan”).

While these systems cover many areas of preventative care, such as preliminary diagnosis and warnings of oral health issues, treatment planning, consultation suggestions, and even follow-up options, they still fall short on providing actionable, personalized recommendations of actual oral care products or ingredients to be used for addressing any identified oral health issues.

In practice, people tend to use generally recommended or heavily advertised oral care means, such as toothbrushes, toothpastes, and dental floss products, without considering the actual features of their own oral cavities, such as existing dental issues, saliva composition, or recorded dental history. Some people may use prescribed oral care means with specific ingredients targeting an identified dental problem, but that usually happens at a stage where a dental professional already had to be involved to manually make a diagnosis and decide on a recommended product or ingredient.

Some prior art solutions may include sensor-based analysis of the dental health of a user of a particular product. One example is EP3327602B1 wherein sensors of an electric toothbrush are used to detect physical dimensions of the oral cavity and to suggest a particular oral care implement or dentifrice. However, this solution requires the use of a specific toothbrush and is limited to identifying physical dental features and recommending specific types of oral care means for addressing such features.

Further prior art solutions such as EP2043509B1 include locally deployed point-of-sale kiosks that would suggest specific oral care products for consumers using the kiosks based on pixel color analysis of an image taken of a consumer's gingival tissue. However, this solution is not available for remote users, and focuses only on a specific region of the oral cavity and a limited capacity image analysis thereof, thus not suitable for providing oral care means that take into account more complex features of the oral cavity.

Accordingly, there is a need for solutions that can provide remote users with customized recommendations of oral care ingredients and/or customized oral care products based on the current state of their oral cavity and additional anamnestic information that the users can provide using existing tools, such as a mobile device that is connected to the Internet and comprises an input interface (e.g. touchscreen) and a camera, without requiring medical expertise or training, without the need to buy specific tools, or to travel to a specific location or consulting a dental professional.

Furthermore, the recommended ingredients and/or products must be able to address the most common conditions of the different regions of the oral cavity including both the teeth, the gums, and the lips.

### SUMMARY

It is an object to provide a method and corresponding computer program product that fulfills these needs and thereby overcomes or at least reduces the problems mentioned above. The foregoing and other objects are achieved by the features of the independent claims. Further implementation forms are apparent from the dependent claims, the description, and the figures.

According to a first aspect, there is provided a computer-implemented method for providing customized oral care means for a person, the method comprising obtaining at least one digital image of an oral cavity of a person, using a camera of a mobile device; obtaining non-image data comprising anamnestic information associated with the person, using the mobile device; processing the at least one digital image using a statistical algorithm trained to identify dental features present in an oral cavity based on digital images of a plurality of oral cavities; assigning an oral cavity profile to the person based on the identified dental features and the non-image data; and determining at least one oral care means based on the assigned oral cavity profile.

The method described can provide users with quick, easy and remote access to recommendations of custom-selected or manufactured oral care products, or recommendations of oral care ingredients or their specific combinations. The recommendations are based on the current state of their oral cavity and can further take into account medical history, both of which the users can provide using a simple mobile device that is connected to the Internet and comprises an input interface (e.g. touchscreen) and a camera, without requiring medical expertise or training.

Due to the steps of analysis of the different types of inputs, and the fact that users are assigned a unique oral cavity profile, the recommended ingredients and/or products can address the conditions of the different regions of the oral cavity including both the teeth, the gums, and the lips.

In a possible embodiment at least one image for each of a set of predefined priority views of the user's oral cavity is obtained.

In a possible embodiment obtaining the at least one digital image further comprises obtaining indication of region of interest for at least one digital image, and wherein processing the at least one digital image using a statistical algorithm takes into account the indication of region of interest for identifying dental features present in the region of interest.

In a possible embodiment obtaining the at least one digital image 1 comprises obtaining a sequence of digital images 1 in the form of a video of the oral cavity 31.

In a possible embodiment at least one oral cavity profile is associated with a respective oral feature map comprising at least one dental feature mapped to specific regions of the oral cavity.

In a possible embodiment the statistical algorithm is a statistical image recognition algorithm that uses a neural network model, more preferably a deep neural network model. In a further possible embodiment, the statistical image recognition algorithm uses a VGG architecture or variants of this architecture, such as a VGG16 architecture.

In another possible embodiment the statistical algorithm is a statistical object detection algorithm that uses a neural network model, more preferably a convolutional neural network model. In a possible embodiment the statistical object detection algorithm uses an R-CNN model or variants of this architecture. In a further possible embodiment, the statistical object detection algorithm uses a Faster RCNN model or variants of this architecture. In a further possible embodiment, the statistical object detection algorithm uses a Mask R-CNN model or variants of this architecture. In a further possible embodiment, the statistical object detection algorithm uses ResNet or variants of this architecture. In a further possible embodiment, the statistical object detection algorithm uses a YOLO (You Only Look Once) model or variants of this architecture.

In a possible embodiment the at least one digital image and the non-image data are processed locally on the mobile device, using at least one processor of the mobile device. In another possible embodiment, the at least one digital image and the non-image data are transmitted to a remote server and processed on the remote server.

In a possible embodiment the non-image data comprises self-reported user input obtained using the mobile device in the form of structured responses given in response to predefined response options primarily centered around medical history and user preferences, in the form of at least one of a checklist, or a slider bar.

In a possible embodiment the non-image data comprises self-reported user input obtained using the mobile device in the form of unstructured responses given in the form of free text, to be used for keyword extraction and sentiment analysis of how affected the person seems to be.

In a possible embodiment the method further comprises processing e data using a syntax analysis algorithm to extract a structured database of non-image signals to be further used for identifying dental features, assigning oral cavity profiles, or determining oral care means, the non-image signals indicating at least one of
symptoms or signs of health regarding intraoral colors, surface structures, contours, wounds, texture, general health, taste in mouth, breath, saliva, healing process, pain;
symptom triggers related to touch or icing, biting down, movement of tongue or jaw;
preferences regarding certain oral care products or ingredients, or
behavioral data regarding oral habits, general health, medicine intake, comorbidities of periodontitis, history of trauma, history of oral procedures, pregnancy, or tobacco usage.

In a possible implementation form of the first aspect determining the oral cavity profile to be assigned to a person is based on a profile framework comprising a plurality of oral cavity profiles, each oral cavity profile being associated with at least one dental feature and optional anamnestic information.

In a possible implementation form of the first aspect the method further comprises:
obtaining a saliva sample from the person;
performing genome analysis on the saliva sample in a remote facility for determining a saliva profile comprising information about at least one of bacterial species and phylotypes; and
assigning an oral cavity profile to the person further based on the saliva profile.

Examples of saliva profiles may include: “mouth is susceptible to dry mouth”, or “saliva has high mineralization of plaque”.

In a possible implementation form of the first aspect the identified dental features comprise a feature score indicating at least one of an amount, likelihood, or severity of a respective dental feature on a numerical scale, and the profile framework comprises oral cavity profiles associated with respective feature scores of dental features.

Examples for feature scores comprise: “visual signs of dental caries on scale 0-3”, “inflammation of gums on scale 0-3”, “crowding of teeth on scale 0-3”, “posterior open bite on scale 0-2”, etc.

In a possible implementation form of the first aspect determining the at least one oral care means comprises performing a severity threshold check for a set of priority dental features according to a conditional sequence based on the effect of the respective priority dental features on the oral health of the person, wherein each step of the conditional sequence comprises comparing the identified feature score of the priority dental feature for the person to a predefined severity threshold value; wherein if the condition is fulfilled (i.e. the feature score equals or is more than severity threshold value) an oral care means defined in the respective step is selected for the person.

In a possible embodiment the obtained non-image data further comprises selection of a topic by the person, and wherein selecting the set of priority dental features is based on the selected topic.

In a possible embodiment the oral cavity profile comprises an oral health assessment such as “dental plaque”, “distribution of dental plaque on teeth”, or “sugar-density in mouth/spit”; and/or detected presence an oral health finding such as “gingivitis”, “periodontitis”, “dental cavities”, or “herpes labialis”.

In a possible embodiment the identified dental features may comprise and may trigger assignment of oral health findings of:
“swelling gums”, “bleeding gums”, “redness of gums”, “dental plaque” may trigger assignment of oral health finding of “gingivitis”;
“exposure of root”, “exposure of dentin”, “abrasion of tooth”, “staining of tooth”, “recession of gums” may trigger assignment of oral health finding of “periodontitis”;
“cavities catalyzing bacteria”, “exposure of decayed dentin”, “density of enamel”, “white spot lesion”, “brown/black discoloration of tooth”, “enamel breakdown”, “shade on enamel” may trigger assignment of oral health finding of “dental caries”;
“inflamed papules and vesicles”, “fluid-filled blister”, “local redness of lip”, “open ulcer” may trigger assignment of oral health finding of “herpes labialis”; and
“light coral pink gums”, “stippled surface gums”, “tightly fitting gumline on tooth” may trigger assignment of oral health finding of “healthy gums”.

In a possible implementation form of the first aspect determining the at least one oral care means based on the assigned oral cavity profile comprises using a decision-tree logic based on dental features and optionally respective feature scores associated with the oral cavity profile.

In another possible implementation form of the first aspect determining the at least one oral care means based on the assigned oral cavity profile comprises analyzing dental features and optionally respective feature scores associated with the oral cavity profile using regression analysis where persons are mapped into sub-populations in a statistical space of predefined dimensions, wherein persons with similar oral cavity profiles are part of a same sub-population, and likely to benefit from oral care means that have been statistically effective for the sub-population.

In another possible implementation form of the first aspect determining the at least one oral care means based on the assigned oral cavity profile comprises analyzing the oral cavity profile using a machine learning algorithm trained to identify oral care means likely to be effective based on associated information with the oral cavity profile, the associated information comprising dental features and optionally respective feature scores, non-image data, and optionally follow-up user input.

In a possible embodiment the machine learning algorithm uses a neural network model.

In a possible embodiment the follow-up user input comprises follow-up image and non-image input obtained after a period of use of a determined oral care means.

In a possible implementation form of the first aspect the at least one oral care means comprises at least one oral care ingredient statistically associated with an effect on the identified dental features present in the oral cavity.

In a possible embodiment the effect on the identified dental features is a negative effect, wherein the determined oral care means will be provided for the person as a contraindication, i.e. oral care means to avoid based on the identified dental features.

In another possible implementation form of the first aspect the at least one oral care means comprises an ingredient combination comprising a plurality of oral care ingredients according to specific proportions or amounts, the ingredient combination being statistically associated with an effect on the identified dental features present in the oral cavity.

In a possible embodiment a relational logic is applied between the plurality of oral care ingredients defining combined effects of any oral care ingredient when used together, wherein the combined effect may be a compound effect, a counter-effect, or a legal effect.

In another possible implementation form of the first aspect the at least one oral care means comprises an oral care product with at least one mechanical, biological, or chemical characteristic statistically associated with an effect on the identified dental features present in the oral cavity.

In a possible embodiment the oral care product comprises at least one oral care ingredient or an ingredient combination according to specific proportions or amounts statistically associated with an effect on the identified dental features present in the oral cavity.

In a possible implementation form of the first aspect determining the at least one oral care means for an oral cavity profile is further based on a treatment framework comprising a plurality of oral care means, each oral care means being associated with at least one oral cavity profile, based on effectiveness of the oral care means as possible treatment in respect of dental features associated with respective oral cavity profiles.

In a possible embodiment the treatment framework comprises routine information for the oral care means when associated with an oral cavity profile, defining an optimal frequency of use of the oral care means that is statistically associated with an effect on the identified dental features present in the oral cavity; and the determined oral care means further comprises the associated routine information for the oral cavity profile of the person.

In a possible embodiment determining the at least one oral care means for an oral cavity profile is further based on an eligibility framework comprising eligibility information of a plurality of oral care means, each oral care means being associated with at least one eligible dental feature and optionally respective feature scores of eligible dental features defining eligibility for recommending the oral care means for a person with an oral cavity profile associated with the at least eligible one dental feature.

In a possible embodiment determining the at least one oral care means for an oral cavity profile is further based on a preference framework comprising preference information of at least one oral care means associated with a respective person. The preference information is not only related to banal statements “I like this”, but motivated by how enjoyable treatment is for a person and has an effect on compliance and ultimately medical effectiveness.

In a possible embodiment the oral care means are determined locally on the mobile device using at least one processor of the mobile device. In a further possible embodiment, the oral care means are determined on a remote server based on extracted data from the digital images and the non-image data, which may be transmitted thereon via a computer network. In a further possible embodiment, the oral care means are transmitted from the remote server for displaying to the person on the display of the mobile device.

In a possible embodiment the method further comprises:
processing the dental features using a specialist reference database and flagging any dental features defined in the specialist reference database as any one of
   extraordinarily rare,
   extraordinarily malignant, or
   transcending into the domain of general health; and
if a concept is flagged, forwarding it to a human dental reviewer for validation; and
validating the remaining dental features using a validation algorithm arranged for automatic dental feature validation based on a predefined set of validation rules.

In a possible implementation form of the first aspect the method further comprises presenting the at least one oral care means on a display of the mobile device, along with a first match score based on the effectiveness of the at least one oral care means as possible treatment in respect of dental features associated with the oral cavity profile.

In a possible implementation form of the first aspect the method further comprises:
obtaining information related to oral care means currently applied by the person;
determining a second match score based on the effectiveness of the currently applied oral care means as possible treatment in respect of dental features associated with the associated oral cavity profile of the person; and
presenting the second match score on the display of the mobile device.

In a possible implementation form of the first aspect the method further comprises:
presenting the at least one oral care means on a display of the mobile device along with a choice option for the person to “accept” or “reject” the presented oral care means; and
if the person chooses to “reject” the presented oral care means, repeating the steps of the method to obtain newly determined oral care means, starting either from obtaining the at least one digital image, obtaining the non-image data, processing the digital: image, assigning the oral cavity profile, or determining the least one oral care means; with a rule applied that the previously determined and rejected oral care means are excluded from the options to select from as newly determined oral care means.

In a possible implementation form of the first aspect if the person chooses to “accept” a presented oral care means; the method further includes presenting an action screen on the display of the mobile device comprising at least one option for the person to take an action in relation to the oral presented care means.

According to a second aspect, there is provided a computer program product, encoded on a computer-readable storage medium, operable to cause a processor to perform operations according to any possible implementation form of the first aspect.

According to a third aspect, there is provided a method of providing a customized oral care product for a person, the method comprising:
obtaining at least one digital image of an oral cavity of a person and non-image data comprising anamnestic information associated with the person;
determining at least one oral care means for the person based on the at least one digital image and the non-image data, by assigning an oral cavity profile to the person according to any possible implementation form of the first aspect; and
providing a customized oral care product for the person comprising or corresponding to the at least one oral care means.

The method described can provide users with quick, easy and remote access to custom-selected or custom manufactured oral care products based on the current state of their oral cavity and their medical history, both of which the users can provide using a simple mobile device that is connected to the Internet and comprises an input interface (e.g. touchscreen) and a camera, without requiring medical expertise or training.

Due to the steps of analysis of the different types of inputs, and the fact that users are assigned a unique oral cavity profile, the customized oral care products can address the conditions of the different regions of the oral cavity including both the teeth, the gums, and the lips.

In a possible embodiment, in addition to the content of the oral care product, such as ingredient of a toothpaste, the customized oral care product also includes the customization of any emballage such as printing the person's name on the tube's label along with the ingredients.

In an exemplary embodiment the person receives a toothpaste that has been added a specific ingredient and added a name of their own choice to the tube's label.

In a further possible embodiment, other characteristic of an oral care product can also be customized for a person such as labelling, size of oral care product, etc. For example, the name of the person is added to label; or the size of tube is adjusted to user's preference.

In a possible implementation form of the third aspect providing the customized oral care product for a person comprises:
determining a plurality of oral cavity profiles according to any possible implementation form of the first aspect;
selecting a limited set of common oral cavity profiles from the plurality of determined oral cavity profiles;
manufacturing a plurality of Stock Keeping Units, SKUs, each SKU comprising a set of oral care products manufactured for a respective common oral cavity profile;
matching the assigned oral cavity profile of the person with a closest common oral cavity profile; and
providing an oral care product for the person from the SKU manufactured for the closest common oral cavity profile.

In a possible embodiment selecting a limited set of common oral cavity profiles from the plurality of determined oral cavity profiles is based on statistical commonness or occurrence within the plurality of oral cavity profiles.

In a possible implementation form of the third aspect providing the customized oral care product for a person comprises manufacturing an oral care product comprising an oral care ingredient statistically associated with an effect on identified dental features present in the oral cavity of the person.

In a possible implementation form of the third aspect providing the customized oral care product for a person comprises manufacturing an oral care product comprising an ingredient combination comprising a plurality of oral care ingredients according to specific proportions or amounts, the ingredient combination being statistically associated with an effect on identified dental features present in the oral cavity of the person.

In a possible implementation form of the third aspect providing the customized oral care product for a person comprises manufacturing an oral care product with at least one mechanical, biological, or chemical characteristic statistically associated with an effect on identified dental features present in the oral cavity of the person.

In a further possible implementation form of the third aspect providing the customized oral care product comprises shipping or collecting an existing oral care product to the person.

In a possible implementation form of the third aspect the method further comprises:
obtaining feedback from the person on the effectiveness of the provided customized oral care product on identified dental features present in the oral cavity of the person;
determining at least one adjusted oral care means for the person, based on both the assigned oral cavity profile of the person and the feedback; and
providing an adjusted oral care product for the person comprising or corresponding to the at least one adjusted oral care means.

In a possible implementation form of the third aspect obtaining the feedback comprises obtaining follow-up non-image data comprising at least one of follow-up anamnestic information associated with the person covering a period of use of the customized oral care product, or perceptive information regarding the perceived effectiveness of the customized oral care product after a period of use; wherein determining the at least one adjusted oral care means comprises at least one of comparing the originally obtained anamnestic information with the follow-up anamnestic information, or analyzing the perceptive information.

In a possible implementation form of the third aspect obtaining the feedback comprises:
obtaining at least one follow-up digital image of the oral cavity of the person; and
processing the at least one follow-up digital image using a statistical algorithm trained to identify dental features present in an oral cavity based on digital images of a plurality of oral cavities, to identify at least one follow-up dental feature;
wherein determining the at least one adjusted oral care means comprises comparing the originally identified dental features present in the oral cavity of the person to the identified follow-up dental features.

In a possible embodiment obtaining the feedback comprises obtaining dental professional assessment of the oral cavity of the person after a period of use of the customized oral care product.

In a possible implementation form of the third aspect obtaining the feedback comprises obtaining a plurality of feedback from a plurality of persons, and selecting relevant feedback for a user based on statistical similarities between the user and the plurality of persons oral cavity profiles.

In a possible implementation form of the third aspect the customized oral care product comprises associated routine information based on the oral cavity profile of the person; and providing the adjusted oral care product for the person comprises:
determining adjusted routine information based on both the oral cavity profile and the feedback; and
providing the adjusted routine information for the person with the customized oral care product or the adjusted oral care product.

In a possible embodiment the adjusted oral care means comprises supplementary oral care means determined for the person based on the feedback.

These and other aspects will be apparent from and the embodiment(s) described below.

### BRIEF DESCRIPTION OF THE DRAWINGS

In the following detailed portion of the present disclosure, the aspects, embodiments, and implementations will be explained in more detail with reference to the example embodiments shown in the drawings, in which:

FIG. 1 shows a flow diagram of a method for providing customized oral care means for a person in accordance with the first aspect;

FIG. 2 shows a flow diagram of assigning an oral cavity profile to a person in accordance with a possible implementation form of the first aspect;

FIG. 3 shows a flow diagram of assigning an oral cavity profile to a person in accordance with another possible implementation form of the first aspect;

FIG. 4 shows a flow diagram of performing a severity threshold check in accordance with a possible implementation form of the first aspect;

FIG. 5 shows a flow diagram of determining oral care means based on an assigned oral cavity profile in accordance with a possible implementation form of the first aspect;

FIG. 6 shows a flow diagram of determining oral care means based on an assigned oral cavity profile in accordance with another possible implementation form of the first aspect;

FIG. 7 shows a flow diagram of determining oral care means based on an assigned oral cavity profile in accordance with another possible implementation form of the first aspect;

FIG. 8 shows a diagram of customized oral care means in accordance with a possible implementation form of the first aspect;

FIG. 9 shows a flow diagram of determining oral care means and associated routine information in accordance with a possible implementation form of the first aspect;

FIG. 10 shows an illustration of display of a mobile device with match scores displayed along oral care means in accordance with a possible implementation form of the second aspect;

FIG. 11 shows illustrations of displays of a mobile device with choice options displayed along oral care means and their actions in accordance with a possible implementation form of the second aspect;

FIG. 12 shows an illustrations of a specific example of a framework combination for determining oral care means in accordance with a possible implementation form of the first aspect;

FIG. 13 is a block diagram of a system for providing customized oral care means for a person in accordance with a possible implementation form of the second aspect;

FIG. 14 is a diagrammatic representation of a wireless system in accordance with a possible implementation form of the second aspect;

FIG. 15 shows a flow diagram of providing a customized oral care product in accordance with a possible implementation form of the third aspect;

FIG. 16 shows a flow diagram of providing a customized oral care product in accordance with another possible implementation form of the third aspect;

FIG. 17 shows a flow diagram of providing an adjusted oral care product in accordance with a possible implementation form of the third aspect; and

FIG. 18 shows a flow diagram of providing an adjusted oral care product in accordance with another possible implementation form of the third aspect.

### DETAILED DESCRIPTION

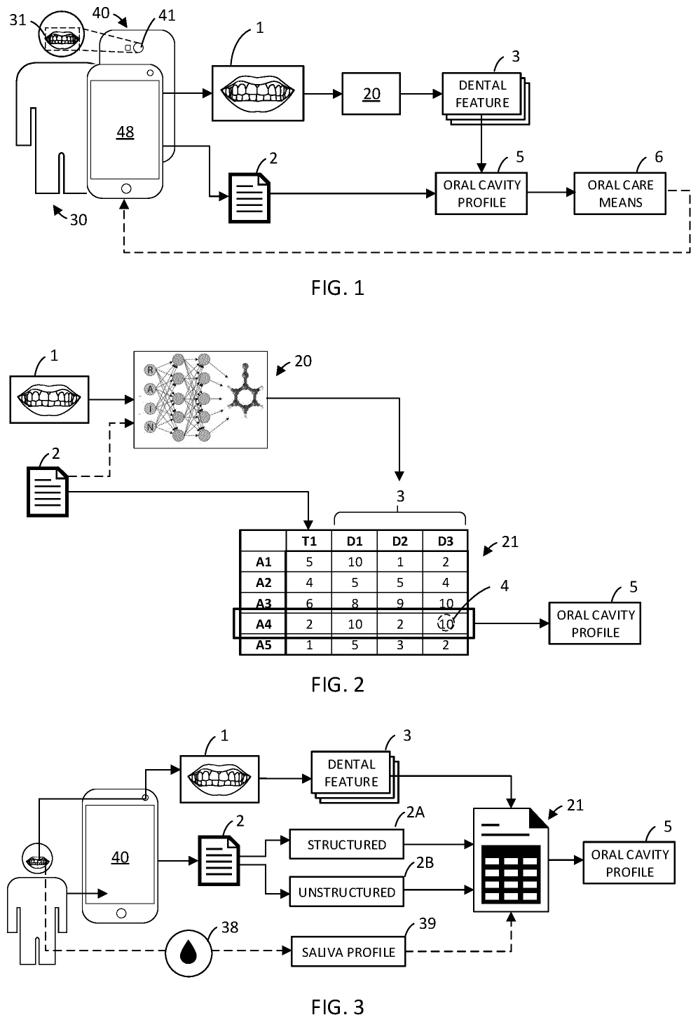

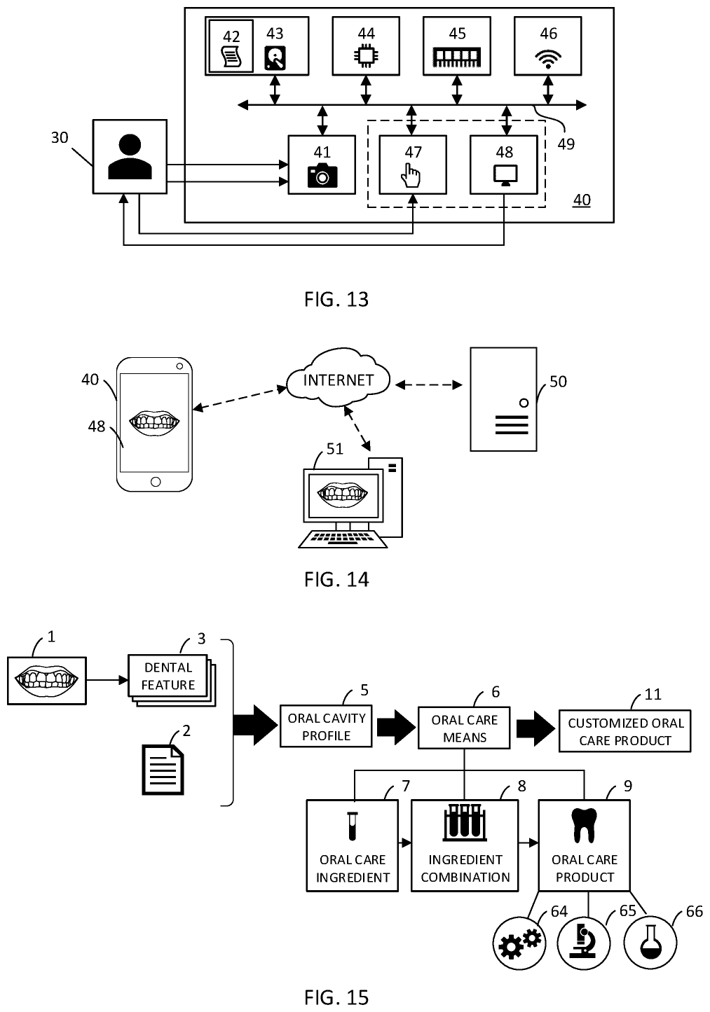

FIG. 1 shows a flow diagram of a method for providing customized oral care means for a person in accordance with the present disclosure, using a computer-based system such as for example the system shown on FIG. 13.

A mobile device 40 is provided, as part of the computer-based system, comprising at least one camera 41 configured to capture digital images and means for obtaining further non-image input. The mobile device 40 is a portable computing device. In the embodiment illustrated in FIG. 1, the mobile device 40 is a smartphone, comprising both a front camera above the display 48 and a main camera 41 on the rear side. In another embodiment, the mobile device 40 may be e.g. a tablet computer (tablet) with corresponding features (display and camera).

In an embodiment, the mobile device 40 is configured to execute an application software (“app”) and comprises a camera 41 for capturing images and a display 48 for displaying the images (as part of a user interface), wherein the display 48 and the camera 41 are provided on opposite sides of the housing of the mobile device 40. In another embodiment, the camera 41 for capturing images may be a secondary camera provided on the same side of the housing of the mobile device as the display 48. In an embodiment the display 48 may comprise a touch screen that provides the means for obtaining non-image input from the user through user interaction with a graphical user interface (GUI).

In an initial step, at least one digital image 1 of the oral cavity 31 of the person 30 using the mobile device 40 is obtained using a camera 41 of the mobile device 40.

Herein, “oral cavity” may refer to e.g. lips, hard palate, soft palate, retromolar trigone (area behind the wisdom teeth), tongue, gingiva (gums), buccal mucosa, the floor of the mouth under the tongue, and/or teeth.

Obtaining the at least one digital image 1 also covers obtaining a series of digital images 1 in the form of a video of the oral cavity 31.

The person 30 may capture the digital image(s) 1 with the camera 41 of the mobile device 40 and/or may add existing images from a gallery available e.g. on or via the mobile device 40 that were taken with the camera 41 beforehand. The obtained digital image(s) 1 may be both an intraoral or extraoral high resolution color photograph(s), preferably in the RGB or RGBA color space.

For providing the at least one digital image 1, the person 30 may be prompted to select a particular view/pose annotation describing the view/pose of the images, e.g. closed mouth view, bite view, bottom/lower arch view, upper arch view, bottom/lower lip view, upper lip view, closed bite anterior view, open bite anterior view, closed bite buccal view, open bite buccal view, roof of mouth view, floor of mouth view, side of mouth view, or frontal view, and may be guided to capture an image in the selected particular view/pose. In an embodiment the person 30 may be required to provide at least one image for each of a set of predefined priority views, such as a ‘frontal view with closed bite’, a ‘bottom lip pulled down, exposing teeth in lower mouth and the inner bottom lip’ and ‘top lip pulled up, exposing teeth in top mouth and the inner top lip’.

In an embodiment, the person 30 whose oral cavity 31 is captured on image is the same person as the person operating the mobile device 40 for obtaining the one or more digital images 1. In another embodiment, the person operating the mobile device 40 is not the same person as the person whose oral cavity 31 is captured on image. This may, for example, be the case when the person 30 whose oral cavity 31 is captured on image is a child or other person requiring help, e.g. from a parent or other family member, for capturing the one or more images of an area of his/her oral cavity.

In a further step, non-image data 2 associated with the person 30 is also obtained using the mobile device 40. The non-image data 2 comprises anamnestic information about the person 30, wherein “anamnestic information” may refer to any type of information regarding the patient's medical history as well as any current symptoms (see described below in detail).

Herein, the numbering of steps does not correspond to a strict order of execution—obtaining the non-image data 2 can happen in the same time, before and/or after obtaining the digital image(s) 1.

In an embodiment the non-image data 2 comprises self-reported user input given by the person via e.g. a touchscreen interface of the mobile device 40. In an embodiment the input may be obtained in the form of a dialogue, where the person 30 answers an automatically generated or predefined sequence of questions and the answers are recorded on the mobile device 40. In an embodiment the questions may be received, and the answers may be given in the form of a checklist, a slider bar, a visual representation, and/or free text through a touchscreen, or as spoken text. In an embodiment, a 3D representation of an oral cavity may be presented to the person 30 for indicating in the representation an area corresponding to the area of the person's own oral cavity 31 associated with an oral health problem. The area may e.g. be a specific tooth. The person's answers may be finite answers.

For example, the person 30 may select one or more suggestions from a checklist.

In an embodiment the sequence of questions to the person 30 may comprise questions relating to past and present behavioral data (such as tobacco use and oral hygiene habits), symptoms, symptom triggers, and/or temporal contextual variables such as urgency. In an embodiment the symptoms may be symptoms of at least one of gingivitis, periodontitis, dental caries, abrasion of tooth, bruxism, cold sore, erosion of teeth, fluorosis, herpes labialis, herpes zoster, or herpes infection.

In a preferred embodiment, the person is presented with a text-based dialogue on the display 48 of the mobile device 40, the dialogue comprising a sequence of questions arranged to guide the person through a process of combined input of both non-image data 2 and digital image(s) 1 in one flow.

Once obtained, the digital images 1 are processed using one or more statistical algorithms 20 trained to identify dental features 3 present in an oral cavity 31. This step is described in detail in the applicant's prior application DK201970623/WO2021064114A1 (“Method for assessing oral health using a mobile device”), which is referenced herein in its entirety, wherein dental features 3 in the present context correspond to local visual features as well as global classification labels obtained according to the prior application.

Accordingly, in a possible embodiment the statistical algorithm 20 is an image recognition algorithm that uses a neural network model, more preferably a deep neural network model. The image recognition algorithm may use a VGG architecture or variants of this architecture, such as a VGG16 architecture.

In another possible embodiment the statistical algorithm 20 is an object detection algorithm that uses a neural network model, more preferably a convolutional neural network (CNN) model.

In an exemplary embodiment the statistical algorithm 20 may use an R-CNN model (and variants of this architecture), wherein the “R” refers to any extracted local feature 3 being associated with a Region (sub-region) of the input image 1. In the R-CNN model the CNN is forced to focus on a single region of the input image 1 at a time to minimize interference. Thus, it is expected that only a single object of interest will dominate in a given region. The regions in the R-CNN are detected by selective search algorithm followed by resizing, so that the regions are of equal size before they are fed to a CNN for classification and bounding box regression. The output of this model is thus at least one local visual feature with a corresponding bounding box and likelihood.

In another exemplary embodiment, the statistical algorithm 20 may use a Faster model (and variants of this architecture) for bounding box object detection of local visual features as described above. While the above algorithms (R-CNN, and even its faster implementation Fast R-CNN) use selective search to find out the region proposals, which is a slow and time-consuming process affecting the performance of the network, the Faster RCNN model is an object detection algorithm that eliminates the selective search algorithm and lets the network learn the region proposals. Similar to R-CNN (or Fast R-CNN), the digital image 1 is provided as an input to a convolutional network which provides a convolutional feature map. However, instead of using selective search algorithm on the feature map to identify the region proposals, a separate network is used to predict the region proposals. The predicted region proposals are then reshaped using a RoI (Region of Interest) pooling layer which is then used to classify the image within the proposed region and predict the offset values for the bounding boxes.

In another exemplary embodiment, the statistical algorithm 20 may (also) use a Mask R-CNN model, which extends the R-CNN model by adding a branch for predicting an object mask in branch for bounding box parallel with the existing recognition. In effect, the processing with a Mask-RCNN model has two stages. The first stage proposes candidate object bounding boxes, while the second stage extracts features using RoI (Region of Interest) Pooling from each candidate box and performs classification, and bounding box/mask regression. The output of this model is thus at least one local visual feature with a corresponding bounding box, object mask and likelihood.

In another exemplary embodiment, the statistical object detection algorithm 20 may (also) use a ResNet (and variants of this architecture), for mask object detection of local visual features as described above. The output of this model (or combination of models) is thus at least one local visual feature with a corresponding bounding box, object mask and likelihood.

In another exemplary embodiment, the statistical algorithm 20 may use a YOLO (You Only Look Once) model (and variants of this architecture) for bounding box object detection of local visual features as described above. YOLO is an object detection algorithm much different from the region-based algorithms described above. In YOLO a single convolutional network predicts the bounding boxes and the class probabilities for these boxes. The output of this model is thus at least one local visual feature with a corresponding bounding box and likelihood.

In an embodiment the statistical algorithm 20 comprises individual binary algorithms optimized for each dental feature 3 respectively, arranged to further predict the likelihood of the dental feature 3, a pointer (such as a boundary box, circle, or point) indicating the location of the dental feature 3, and/or an object mask for the dental feature 3.

A dental feature 3 that corresponds to a local visual feature can be identified through a variety of different annotations, depending on the feature. Annotation types include boundary boxes, polygons, focal points, and individual labelling of intelligently identified subset/local patches of the entire image.

Some examples of dental features 3 relate to local visual features that are indications of gum disease or inflammation such as:
Swelling of gums/type of feature: form, contour, shape/
Redness of gums/type of feature: color, gradient, shade/
Gradient of redness/type of feature: color, gradient, shade/
Bleeding gums
Discolorations of teeth (due to lacking oral hygiene)/type of feature: color, gradient, shade/
External matter on teeth (due to lacking oral hygiene)/type of feature: texture, surface, roughness/
Presence of plaque
Exposure of root/type of feature: (primarily) color, gradient, shade
Recession of gums/type of feature: form, contour, shape/
Cliff separating enamel and dental/type of feature: Form, contour, shape/

Processing the digital images 1 using a statistical image recognition algorithm can further result in global classification labels and their likelihood scores.

In a possible embodiment, the statistical image recognition algorithm uses a VGG architecture, which refers to a very deep neural network that uses little preprocessing of input images, and stacks of convolutional layers, followed by Fully Connected (FC) layers. The convolutional layers in the VGG architecture use filters with a very small receptive field and are therefore optimal for the processing of digital intraoral and extraoral images. The output of this model is thus at least global classification label with a corresponding likelihood value.

In an exemplary embodiment, the statistical image recognition algorithm uses a VGG16 architecture, wherein “16” refers to the number of layers in the architecture.

The extracted global classification labels each correspond to a medical finding related to the user's oral cavity 31 as a whole. Herein, similarly as already defined above, a “medical finding” may refer to both normal and abnormal medical states.

In an embodiment the at least one global classification label corresponds to at least one of “gingivitis” (an inflammatory state of the gums), “active periodontitis” (an inflammatory state of the gums but at a later stage where the gums are detaching from the tooth/dentin), “inactive periodontitis” (a stable, non-inflammatory state of the gums after having long-term/persistent inflammation of the gums that is now under control, i.e. the gums have retracted chronically), “dental cavities” or “caries” (the decay of the tooth, i.e. the bone structure), and/or “herpes labialis” (a type of infection by the herpes simplex virus that affects primarily the lip, also known as “cold sores”).

In a possible embodiment, at least one local visual feature is used as further input for a statistical image recognition algorithm to enhance the predictability of the at least one global classification label.

In a further possible embodiment, the at least one global classification label is used as further input for a statistical object detection algorithm to enhance the predictability of the at least one local visual feature.

In a further embodiment, one or more predefined local visual feature may increase the likelihood of at least one global classification label.

In an embodiment, “swelling gums”, “bleeding gums”, “redness of gums”, “dental plaque” may increase the likelihood of “gingivitis”. In a further embodiment, “exposure of root”, “abrasion of tooth”, “staining of tooth”, “recession of gums” may increase the likelihood of “periodontitis”. In a further embodiment, “white spot lesion”, “brown/black discoloration of tooth”, “enamel breakdown”, “shade on enamel”, “dental plaque” may increase the likelihood of “dental cavities”. In a further embodiment, “inflamed papules and vesicles”, “fluid-filled blister”, “local redness of lip”, “open ulcer” may increase the likelihood of “herpes labialis”.

In a further embodiment, “light coral pink gums”, “stippled surface gums”, “tightly fitting gumline on tooth” may increase the likelihood of “healthy gums”.

In a next step, an oral cavity profile 5 is assigned to the person 30 based on the identified dental features 3 and the non-image data 2, as will be explained below in detail.

Once the oral cavity profile 5 is assigned, in a final step at least one oral care means 6 is determined for the person 30 based on the assigned oral cavity profile 5, as will be explained below in detail.

Similarly as described above with respect to processing the at least one digital image 1 and the non-image data 2, in an embodiment the oral care means 6 can be determined locally on the mobile device 40 using at least one processor 44 of the mobile device 40.

Similarly, in another possible embodiment the oral care means 6 can also be determined a remote server 50 based on the extracted data from the statistical algorithms (the local visual features with their corresponding likelihood, bounding box, and/or object mask, as well as the global classification labels with its corresponding likelihood) and the non-image data 2, which may all be already on the server 50 or can be transmitted thereon using a computer network as shown in FIG. 14. The oral care means 6 can then be transmitted for displaying to the person 30 on the display 48 of the mobile device 40.

FIG. 2 shows a flow diagram of assigning an oral cavity profile 5 to a person 30 in accordance with the present disclosure. In this and further figures, steps and features that are the same or similar to corresponding steps and features previously described or shown are denoted by the same reference numeral as previously used for simplicity. As shown in the figure, determining the oral cavity profile 5 to be assigned to a person 30 can be based on a profile framework 21 comprising a plurality of oral cavity profiles 5, each oral cavity profile 5 being associated with at least one dental feature 3 and optional anamnestic information obtained from the non-image data 2.

The identified dental features 3 may all comprise an associated feature score 4 indicating at least one of an amount, likelihood, or severity of a respective dental feature 3 on a numerical scale, and as shown in FIG. 2, the profile framework 21 may comprise oral cavity profiles 5 associated with respective feature scores 4 of dental features 3.

FIG. 3 shows a flow diagram of assigning an oral cavity profile 5 to a person 30 in accordance with another example of the present disclosure.

As shown in the figure, the method may comprise obtaining a saliva sample 38 from the person 30, performing genome analysis on the saliva sample 38 in a remote facility for determining a saliva profile 39 comprising information about at least one of bacterial species and phylotypes. When there is such a saliva profile 39 associated with a person 30 available, assigning an oral cavity profile 5 to the person 30 is further based on this saliva profile 39.

The non-image data 2 may comprise self-reported user input obtained using the mobile device 40 in the form of structured responses 2A given in response to predefined response options primarily centered around medical history and user preferences, in the form of at least one of a checklist, or a slider bar. For example: “How often do your gums bleed?” “Every day”, “every week”, “less often”.

The non-image data may also comprise self-reported user input in the form of unstructured responses 2B given in the form of free text, to be used for keyword extraction and sentiment analysis of how affected the person seems to be. For example: “Please describe the symptoms you are experiencing”.

The method further may comprise processing the non-image data 2 obtained in the form of structured responses 2A and/or unstructured responses 2B using a syntax analysis algorithm (not shown) to extract a structured database of non-image signals to be further used for identifying dental features 3, assigning oral cavity profiles, or determining oral care means.

The dental features 3, structured responses 2A, unstructured responses 2B, and the optional saliva profile 39 may all be used in the profile framework 21 for assigning the oral cavity profile 5.

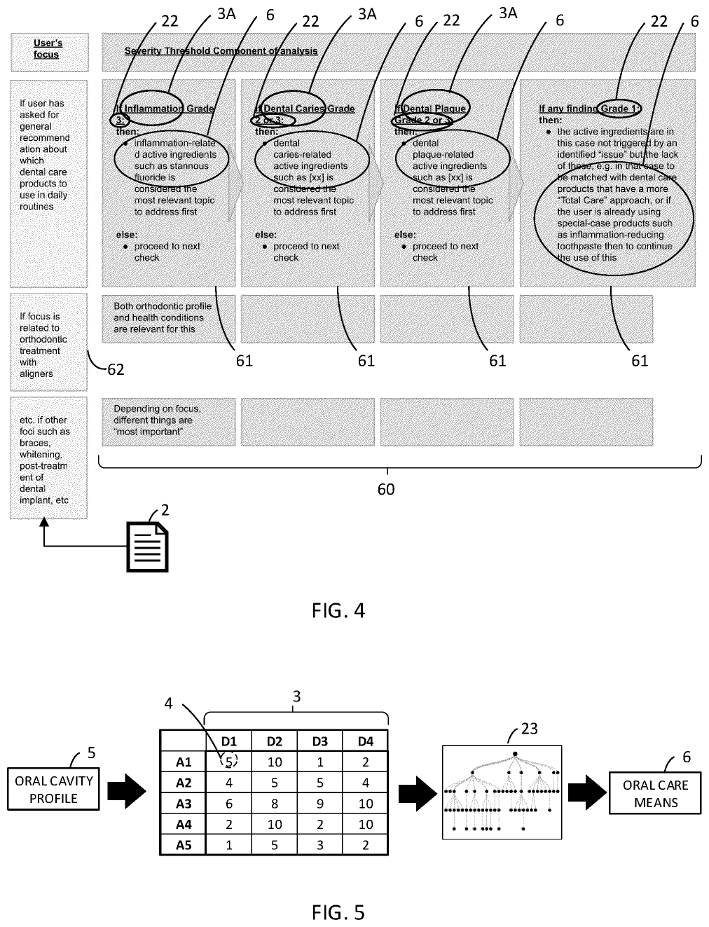

FIG. 4 shows a flow diagram of performing a severity threshold check in accordance with another example of the present disclosure. As illustrated, the method may include a logic for performing a severity threshold check for a set of priority dental features 3A according to a conditional sequence 60. This conditional sequence 60 may be based on the effect of the respective priority dental features 3A on the oral health of the person 30, wherein each step 61 of the conditional sequence 60 comprises instructions for comparing the identified feature score 4 of the respective priority dental feature 3A for the person 30 to a predefined severity threshold value 22; wherein if the condition is fulfilled, e.g. the feature score equals or is more than severity threshold value, an oral care means 6 defined in the respective step is selected for the person.

The conditional sequence may also include a final step 61 wherein oral care means 6 are selected based on a lack of identified priority dental feature 3A that would fulfill any of the preceding conditions. Such final step 61 would generally result in a selection of oral care means 6 based on a “Total Care” approach, or if the person 30 is already using special-case products such as inflammation-reducing toothpaste then to continue the use of this.

The selection of the set of priority dental features 3A may be based on a topic 62 pre-selected by the person 30, wherein obtaining the non-image data 2 comprises a prompt for selection of a topic 62, and wherein the respective conditional sequence 60 differs depending on the selected topic 62.

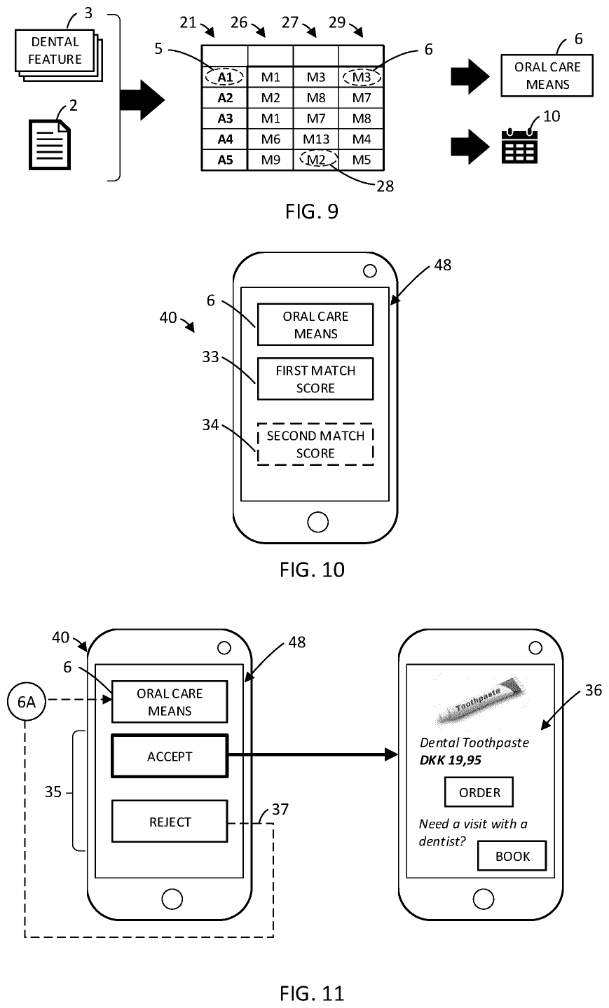

After performing the severity threshold check, determining the oral care means 6 for the oral cavity profile 5 may be further based on an eligibility framework 27 comprising eligibility information of a plurality of oral care means 6, each oral care means 6 being associated with at least one eligible dental feature 28 and optionally respective feature scores 4 of eligible dental features 28 defining eligibility for recommending the oral care means 6 for a person 30 with an oral cavity profile 5 associated with the at least eligible one dental feature 28 (as illustrated in FIG. 9).

For example, a whitening treatment with hydrogen peroxide would require a minimal-strength of the enamel to be effective and recommendable for a person 30 as oral care means 6.

Determining the at least one oral care means 6 for an oral cavity profile 5 may further be based on a preference framework 29 comprising preference information of at least one oral care means 6 associated with a respective person 30. The preference information is obtained as part of the non-image data 2, and is not only related to banal statements “I like this”, but motivated by how enjoyable an oral care product is for a person, or what ultimate goal they want to achieve, such as “I am interested in making my teeth less sensitive” or “I like fresh mint in my toothpaste”, which in turn has a positive effect on compliance and ultimately medical effectiveness.

For example, when a person 30 prefers a high degree of foam and mintiness in their toothpaste for them to perceive their use of the product has an effect, it will be taken into account when selecting an oral care product 9 or ingredient 7. Other preference examples may include: flavor of toothpaste; waxed or not-waxed floss; bristle stiffness; rotation types of an electric toothbrush.

Accordingly, steps executed after the severity threshold check may include matching the person 30 with the subset of active ingredients that also match the user's eligibility and preferences such as whether they are vegan, or do not wish to use toothpaste with fluoride as an active component and therefore need an alternative (such as Hydroxyapatite which—despite a lot of non-fluoride toothpaste being popular on the market—is as of 2021 one of the only evidence based alternatives to the positive effects of fluoride effects to teeth).

FIG. 5 shows a flow diagram of determining oral care means 6 based on an assigned oral cavity profile in accordance with another example of the present disclosure. As illustrated, determining the at least one oral care means 6 for the assigned oral cavity profile 5 may be executed using a decision-tree logic 23 based on dental features 3 and optionally respective feature scores 4 associated with the oral cavity 31 profile.

The input modalities in the decision-tree logic may be processed in a “if this, then this treatment may (not) be more relevant” manner. In an exemplary embodiment where gums show visual signs of active inflammation (grade 2 or 3 on a scale to 3) and recession of gums show signs of this being a persistent state of inflammation, it is determined likely that a dental care product that has long-term effect on gum inflammation may be more suitable than a product that addresses short term inflammation.

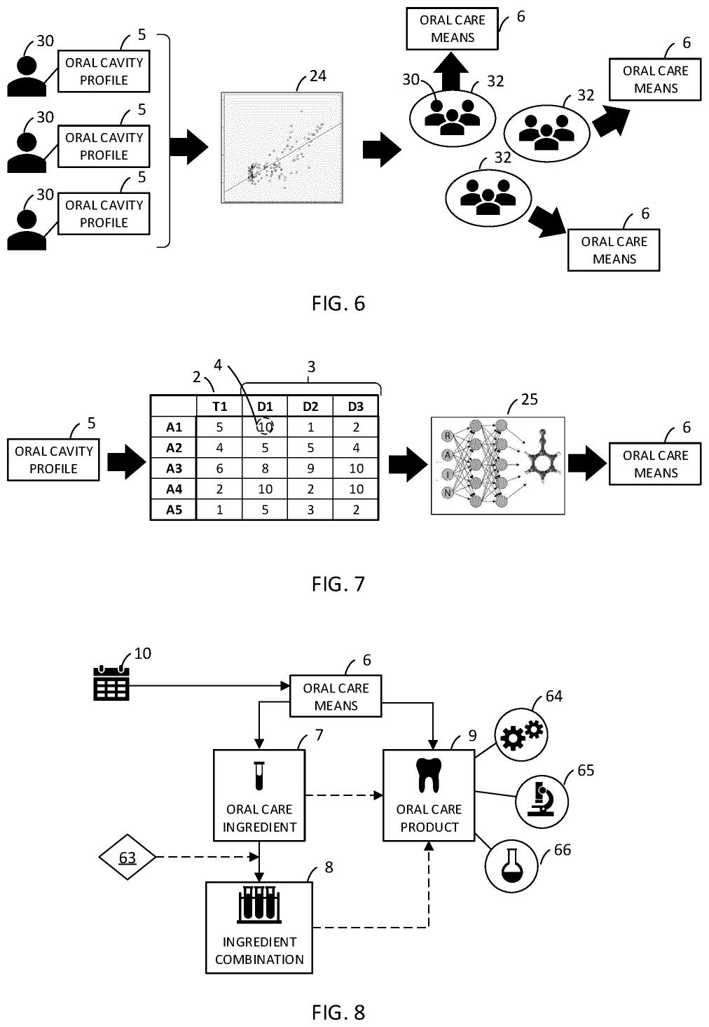

FIG. 6 shows a flow diagram of determining oral care means 6 based on an assigned oral cavity profile in accordance with another example of the present disclosure. In this example determining the oral care means 6 is based on analyzing dental features 3 and optionally respective feature scores 4 associated with the oral cavity profile 5 using regression analysis 24 where persons 30 are mapped into sub-populations 32 in a statistical space of predefined dimensions, wherein persons 30 with similar oral cavity profiles 5 are part of a same sub-population 32, and likely to benefit from oral care means 6 that have been statistically effective for the sub-population 32.

For example, if a person 30 has grade 2 dental caries, grade 1 inflammations, etc., other persons 30 with similar combination of findings are part the same sub-population 32, and likely to benefit from treatment that has been effective for this sub-population 32.

FIG. 7 shows a flow diagram of determining oral care means based on an assigned oral cavity profile in accordance with another example of the present disclosure. In this example determining the oral care means 6 is based on analyzing the oral cavity profile 5 using a machine learning algorithm 25 trained to identify oral care means 6 likely to be effective based on associated information with the oral cavity profile 5, the associated information comprising dental features 3 and optionally respective feature scores 4, non-image data 2, and optionally follow-up user input.

In a possible embodiment the machine learning algorithm 25 uses a neural network model, and/or the follow-up user input comprises follow-up image and non-image input obtained after a period of use of a determined oral care means (as will be explained below).

FIG. 8 shows a diagram of customized oral care means 6 in accordance with another example of the present disclosure. As the figure shows, the oral care means 6 may comprise at least one oral care ingredient 7 statistically associated with an effect on the identified dental features 3 present in the oral cavity 31.

This effect on the identified dental features may be a positive effect but also a negative effect, wherein the determined oral care means 6 will be provided for the person 30 as a contraindication, i.e. oral care means 6 to avoid based on the identified dental features 3.

The oral care means 6 may accordingly be presented on the display 48 and comprise descriptions such as “the primary active ingredient in the customized toothpaste will be stannous flour” or “the primary active ingredients in the toothpaste tablets will be hydroxyapatite and hydrogen peroxide”.

As also shown in the figure, the care means 6 may also comprise a specific ingredient combination 8 comprising a plurality of oral care ingredients 7 according to specific proportions or amounts, the ingredient combination 8 being statistically associated with an effect on the identified dental features 3 present in the oral cavity 31.

Herein, a relational logic 63 may also be applied between the plurality of oral care ingredients 7 defining combined effects of any oral care ingredient 7 when used together, wherein the combined effect may be a compound effect, a counter-effect, or a legal effect e.g. concentration allowed without prescription in individual products, for example different types of fluoride in a toothpaste in certain countries may not exceed a total of 1450 ppm.

As also shown in the figure, the care means 6 may also comprise an oral care product 9 with at least one mechanical 64, biological 65, or chemical 66 characteristic statistically associated with an effect on the identified dental features 3 present in the oral cavity 31.

Mechanical characteristics may cover, e.g. in case of an electrical toothbrush, variations of bristle stiffness (hard, medium, soft, super-soft), rotation-speed, or bristle compactness (number of bristles pr. mm); or in case of dental floss variations of thread thickness, or being waxed or non-waxed.

Biological characteristics may cover presence (or non-presence) of biological ingredients such as proteins and enzymes.

Chemical characteristics may cover presence (or non-presence) of chemical ingredients such as a toothpaste with variations of “concentration of fluoride, PPM”, “concentration of triclosan”, or “concentration of Stannous fluoride”.

The oral care product 9, similarly as above, may comprise an ingredient 7 or ingredient combination 8 according to specific proportions or amounts statistically associated with an effect on the identified dental features 3 present in the oral cavity 31.

As described above, the amount of certain ingredients 7 may be also legally limited in some countries or tied to prescriptions above a certain amount in an oral care product 9. For example, a mouthwash containing 3% (or less) hydrogen peroxide can be purchased without prescription and can be found in over-the-counter stores in some countries, while mouthwash products that rely on carbamide peroxide typically contain 10 percent carbamide peroxide and may only be dispensed by dentists to their patients for use at home.

FIG. 9 shows a flow diagram of determining oral care means 6 and associated routine information 10 in accordance with another example of the present disclosure.

As illustrated, determining the at least one oral care means 6 for the oral cavity profile 5 is based on a treatment framework 26 that is used in combination with the previously defined profile framework 21 for assigning the oral cavity profile 5 for the person 30, and optionally also the previously defined eligibility framework 27 and preference framework 29.

The treatment framework 26 specifically may comprise a plurality of oral care means 6, each oral care means 6 being associated with at least one oral cavity profile 5, based on effectiveness of the oral care means 6 as possible treatment in respect of dental features 3 associated with respective oral cavity profiles 5.

The effectiveness of the oral care means 6 may be based on general dental research and knowledge, or proprietary research and knowledge from internal research on the oral care means 6.

As further illustrated, the treatment framework 26 may also comprise routine information 10 for the oral care means 6 when associated with an oral cavity profile 5, defining an optimal frequency of use of the oral care means 6 that is statistically associated with an effect on the identified dental features 3 present in the oral cavity 31. Accordingly, the determined oral care means 6 may comprise the associated routine information 10 for the oral cavity profile 5 of the person 30.

For example, stannous flour may be selected for oral care means 6 as it has clinically proven effective in toothpaste applied twice a day for persons 30 with increased risk of gum inflammation.

FIG. 10 shows an illustration of display 48 of a mobile device 40 with match scores displayed along oral care means 6 in accordance with another example of the present disclosure. In particular, the method according to this example further comprises presenting the oral care means 6 on the display 48 of the mobile device 40 along with a first match score 33 based on the effectiveness of the at least one oral care means 6 as possible treatment in respect of dental features 3 associated with the oral cavity 31 profile.

In a possible embodiment a plurality of oral care means 6 may be presented on the display 48, based on their first match scores 33, along with a scale rating, or text description of why the match between oral care means 6 and the person 30 is being suggested.

As also illustrated, the method may further comprise obtaining information related to oral care means 6 currently applied by the person 30, and determining a second match score 34 based on the effectiveness of the currently applied oral care means 6 as possible treatment in respect of dental features 3 associated with the associated oral cavity profile 5 of the person 30. Once calculated, this second match score 34 may (also) be presented on the display 48 of the mobile device 40. In an exemplary embodiment presenting the second match score 34 on the display 48 may include a description such as “the recommendation has a 94% match rate with you. In comparison your current toothpaste has a match rate of 59%”.

FIG. 11 shows illustrations of displays 48 of a mobile device 40 with choice options 35 displayed along oral care means 6 and their actions in accordance with another example of the present disclosure.

In particular, the oral care means 6 would be presented on the display 48 of the mobile device 40 along with a choice option 35 for the person 30 to “accept” or “reject” the presented oral care means 6. If the person 30 chooses to “reject” the presented oral care means 6, the steps of the method would be repeated to obtain newly determined oral care means 6A, starting either from obtaining the at least one digital image 1, obtaining the non-image data 2, processing the digital image 1, assigning the oral cavity profile 5, or determining the least one oral care means 6; with a rule applied that the previously determined and rejected oral care means 6 are excluded from the options to select from as newly determined oral care means 6A.

On the other hand, if the person 30 chooses to “accept” the presented oral care means 6 an action screen 36 would be presented on the display 48 of the mobile device 40 comprising at least one option for the person 30 to take an action in relation to the oral presented care means. Such an action would be for example “purchase”, “refill” (triggering refill of stock with updated means), “book” (such as a consultation with a dental professional) or “place pre-order”.

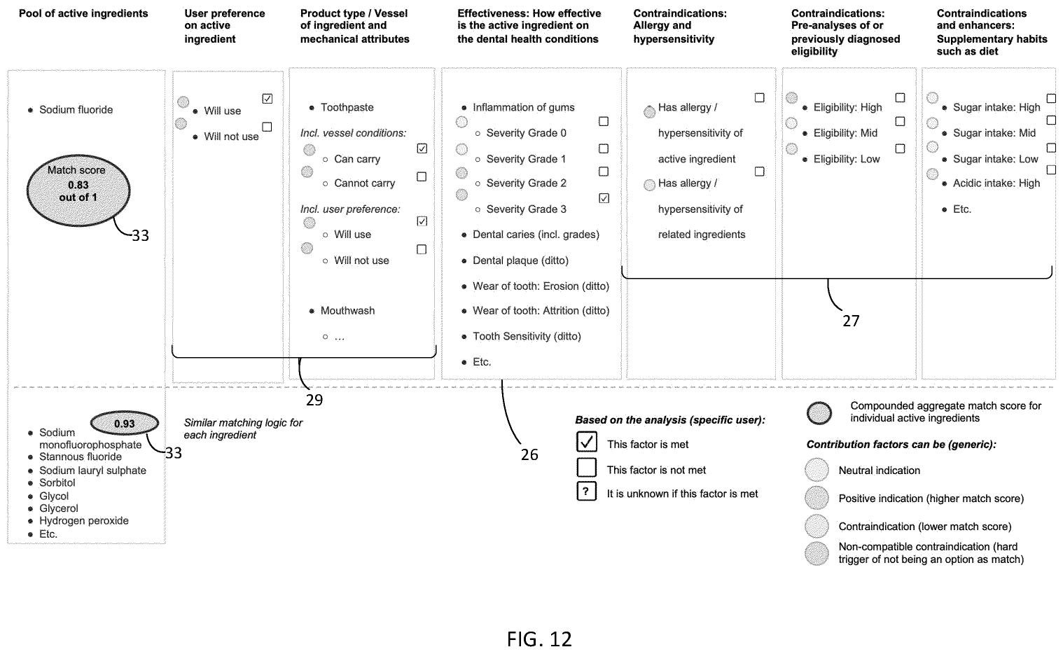

FIG. 12 shows an illustration of a specific example of a framework combination for determining oral care means 6 and their respective match scores in accordance with another example of the present disclosure.

As described already above with respect to FIG. 9, determining the at least one oral care means 6 for the oral cavity profile 5 may be based on a treatment framework 26 that is used in combination with an eligibility framework 27 and preference framework 29. In particular, the exemplary framework illustrated in FIG. 12 shows a list of oral care ingredients 7 and a matching logic for each ingredient 7 using a combination of associated user preferences based on the preference framework 29, associated effectiveness based on the treatment framework 26, and associated eligibility based on the eligibility framework 27. As can be further seen in this exemplary embodiment, the user preferences may relate not only to the oral care ingredient 7, but also to one or more oral care product 9 that may serve as a vessel for the oral care ingredient 7. The associated eligibility may further comprise contraindicators regarding as allergy or hypersensitivity of a respective oral care ingredient 7 or any related ingredients that could be commonly used together with the respective oral care ingredient 7 in an ingredient combination 8 or oral care product 9; contraindicators regarding previously diagnosed eligibility of the person 30; or contraindicators determined based on the obtained non-image data 2, such as sugar intake or acidic intake.

Based on all the above, a first match score 33 is calculated as described before, for display on the mobile device 40.

FIG. 13 is a block diagram of a system for providing customized oral care means 6 for a person 30 in accordance with the present disclosure.

The illustrated mobile device 40 comprises a camera 41 for obtaining at least one digital image 1 of the oral cavity of a person 30, and a machine-readable storage medium 43.

The methods described above may at least partly be implemented as a computer program product 42 encoded on the machine-readable storage medium 43. The computer program product 42 may in effect be realized in the form of an application software (“app”) which may be executed on the mobile device 40 by one or more processors 44 which may load the application software on a memory 45 and result in providing a graphical user interface on a display 48 of the mobile device 40. In an embodiment the display 48 may comprise a touchscreen input interface 47 through which the person 30 may interact with the application software on the mobile device 40.

The mobile device 40 may further comprise an integrated or external communications interface 46 for connecting to other mobile devices or a computer network. For example, the communications interface 46 can include Wi-Fi enabling circuitry that permits wireless communication according to one of the 802.11 standards or a private network. Other wired or wireless protocol standards, such as Bluetooth, can be used in addition or instead.

The mobile device 40 may further comprise an internal bus 49 arranged to provide a data transfer path for transferring data to, from, or between the mentioned components of the mobile device 40.

FIG. 14 is a diagrammatic representation of a wireless system in accordance with the present disclosure. The mobile device 40 described above can communicatively be connected to a network of computer nodes using an integrated or external communications interface 46 as shown in FIG. 11, e.g. for transmitting image data 1 and/or non-image data 2 from the mobile device 40 to a computer node and/or for receiving data from a computer node. The figure shows a diagrammatic representation of an exemplary wireless network, such as a cellular telephone network, the internet, or a cloud-based network, comprising the mobile device 40, a remote server 50, and a dental professional's workstation 51. Image data 1 and/or non-image data 2 may be transmitted from the mobile device 40 to the remote server 50 and/or the dental professional's workstation 51 for remote assessment, processing, validation, and/or storage of the data. The transmission of data may be synchronous, asynchronous, or a mixture of synchronous and asynchronous data transmission.

FIG. 15 shows a flow diagram of providing a customized oral care product 11 in accordance with another example of the present disclosure.

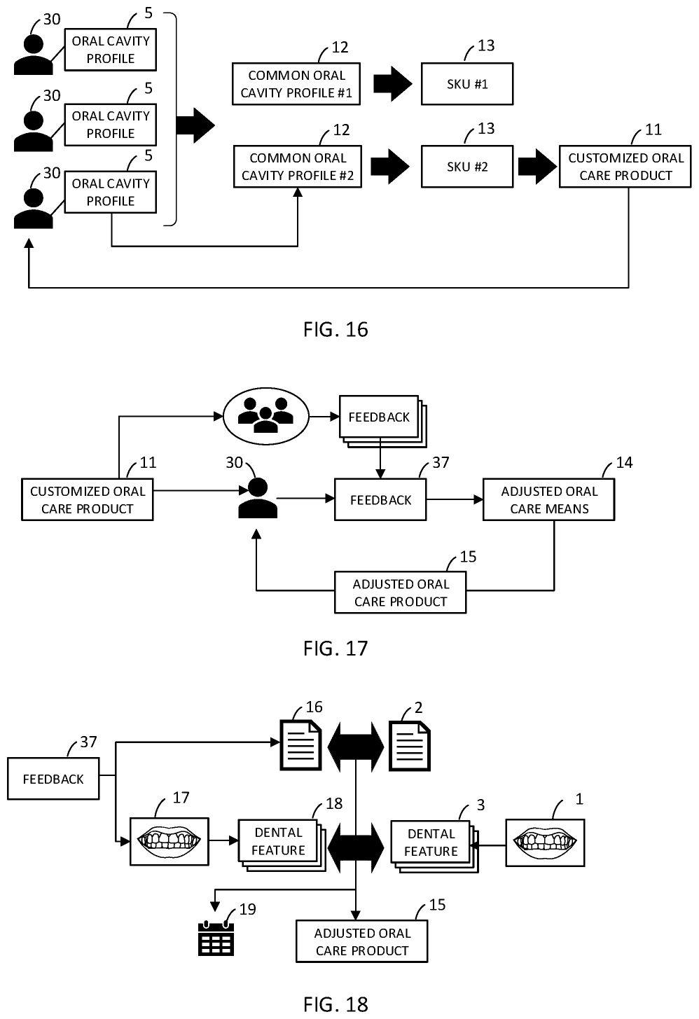

FIG. 16 shows a flow diagram of a method for providing a customized oral care product in accordance with another example of the present disclosure. The method may comprise obtaining digital images 1 of an oral cavity 31 of a person 30, as well as non-image data 2 comprising anamnestic information associated with the person 30, and determining at least one oral care means 6 by assigning an oral cavity profile 5 to the person 30 according to the methods described before. Once the oral care means 6 are determined, a customized oral care product 11 can be provided for the person 30 comprising or corresponding to the oral care means 6. In the simplest example, providing the customized oral care product may comprise collecting and shipping an existing oral care product 9 to the person 30.

In another example, providing the customized oral care product 11 for the person 30 may comprise manufacturing an oral care product 9 comprising an oral care ingredient 7 statistically associated with an effect on identified dental features 3 present in the oral cavity 31 of the person; or manufacturing an oral care product 9 comprising an ingredient combination 8 comprising a plurality of oral care ingredients 7 according to specific proportions or amounts, the ingredient combination 8 being statistically associated with an effect on identified dental features 3 present in the oral cavity 31 of the person. Providing the customized oral care product 11 may also be achieved by manufacturing an oral care product 9 with at least one mechanical, biological, or chemical characteristic statistically associated with an effect on identified dental features 3 present in the oral cavity 31 of the person.

In a possible embodiment, in addition to the content of the oral care product, such as ingredient of a toothpaste, the customized oral care product also includes the customization of any emballage such as printing the person's 30 name on the tube's label along with the ingredients.

For example, the person 30 may receive a toothpaste that has been added a specific ingredient. In further possible embodiments, other characteristics of an oral care product can also be customized for a person 30 such as labelling, size of oral care product, etc. For example, the name of the person 30 may be added to label; or the size of tube is adjusted to person's 30 preference.

In another example illustrated in FIG. 16, providing the customized oral care product 11 for a person 30 comprises determining a plurality of oral cavity profiles 5 according to the methods of any one of claims 1 to 14 and selecting a limited set of common oral 1 cavity profiles 12 from the plurality of determined oral cavity profiles 5, e.g. based on statistical commonness or occurrence within the plurality of oral cavity profiles.

Once common oral cavity profiles 12 are defined, a plurality of Stock Keeping Units, SKUs 13, can be manufactured, each SKU 13 comprising a set of oral care products 9 manufactured for a respective common oral cavity profile 12.

Accordingly, providing the customized oral care product 11 for a person 30 can be achieved by matching the assigned oral cavity profile 5 of the person 30 with a closest common oral cavity profile 12 and providing an oral care product 9 for the person 30 from the SKU 13 manufactured for the closest common oral cavity profile 12, thereby simplifying the process and making manufacture more effective, while still providing sufficient customization for a large majority of people.

FIG. 17 shows a flow diagram of providing an adjusted oral care product 15 in accordance with another example of the present disclosure. In this example, the above described method further comprises obtaining feedback 37 from the person 30 on the effectiveness of the provided customized oral care product 11 on identified dental features 3 present in the oral cavity 31 of the person 30. Based on both the originally assigned oral cavity profile 5 of the person 30 and the feedback 37 at least one adjusted oral care means 14 can be determined for the person 30, and finally an adjusted oral care product 15 can be provided for the person 30 comprising or corresponding to the at least one adjusted oral care means 14.

In an exemplary embodiment when a person 30 sees positive improvement with significantly lower levels of inflammation after week 4, the adjusted oral care product 15 is adapted to the progress by lowering the level inflammation reducing active ingredient in favor of other ingredients.

In a possible embodiment obtaining the feedback 37 may be executed by the person 30 receiving and answering a survey about the effectiveness of the customized oral care product, including the effects they have seen and their own compliance. Alternatively, as also illustrated in FIG. 17, obtaining the feedback 37 may comprise obtaining a plurality of feedback 37 from a plurality of persons 30 (but not the user in question), and selecting relevant feedback 37 user based on statistical similarities between the user and the plurality of persons 30 based on e.g. their respective assigned oral cavity 31 profiles, thereby being able to adjust the oral care means 14 and the oral care product 15 for a person 30 without directly obtaining feedback 37 from them, making the process for certain users quicker and simpler.

As illustrated in FIG. 18, obtaining the feedback 37 may comprise obtaining follow-up non-image data 16 comprising at least one of follow-up anamnestic information associated with the person 30 covering a period of use of the customized oral care product 11, or perceptive information regarding the perceived effectiveness of the customized oral care product 11 after a period of use. In this case, determining the adjusted oral care means 14 comprises either comparing the originally obtained anamnestic information with the follow-up anamnestic information, or analyzing the perceptive information in itself using a syntax analysis method as described before.

Alternatively, or in combination with the above, obtaining the feedback 37 may also comprise obtaining at least one follow-up digital image 17 of the oral cavity 31 of the person 30; and similarly as described before, processing the follow-up digital images 17 using a statistical algorithm 20 trained to identify dental features 3 present in an oral cavity 31, to identify at least one follow-up dental feature 18. In this case, determining the adjusted oral care means 14 comprises comparing the originally identified dental features 3 present in the oral cavity 31 of the person 30 to the identified follow-up dental features 18.

In possible embodiments the person 30 may be asked or granted the opportunity to do a follow-up dental assessment from home, including both uploading intraoral digital images 1 and answering further questions in the form of non-image data 2 for statistical analysis.

In exemplary embodiments the person 30 may be prompted through an app on the mobile device to perform a dental home scan after 4 weeks, 8 weeks and 12 weeks to track any progress related to their oral cavity.

As another alternative or combination, obtaining the feedback 37 may also comprise obtaining dental professional assessment of the oral cavity 31 of the person 30 after a period of use of the customized oral care product 11. In exemplary embodiments the person 30 may be randomly selected for trial study where a dental professional assesses the person's oral cavity 31 in a clinic on week 0, week 4, week 8, week 12, and week 24.

As further illustrated in FIG. 18, and as mentioned before, in certain embodiments the customized oral care product 11 may comprise associated routine information 10 based on the oral cavity profile 5 of the person 30. In such cases, providing the adjusted oral care product 15 for the person 30 may comprise determining adjusted routine information 19 based on both the oral cavity profile 5 and the feedback 37, and providing the adjusted routine information 19 for the person 30 with the customized oral care product 11 or the adjusted oral care product 15, on the display 48 of the mobile device.

In an exemplary embodiment if the person 30 was recommended to use a toothpaste twice a day, but has only complied to doing it once in total, they may be recommended to instead set up a notification to use it once a week to start with to build the habit and then increase the frequency over time.

Furthermore, in possible implementations the adjusted oral care means 14 comprises supplementary oral care means 6 (such as professional dental treatment or additional oral care products to address the issues determined from the obtained feedback) determined for the person 30 based on the feedback 37. In an exemplary embodiment if the person 30 has been recommended using a customized oral care product 11 to address dental plaque and gum care, and there are signs of a natural build-up of tartar, it may be recommended to supplement the treatment with a professional cleaning or additional products to address the build-up.

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems.

The reference signs used in the claims shall not be construed as limiting the scope.

1. A computer-implemented method for providing customized oral care means for a person (30), said method comprising:
obtaining at least one digital image (1) of an oral cavity (31) of a person (30), using a camera (41) of a mobile device (40);
obtaining non-image data (2) comprising anamnestic information associated with said person (30), using said mobile device (40);
processing said at least one digital image using a statistical algorithm trained to identify dental features present in an oral cavity based on digital images of a plurality of oral cavities; wherein said identified dental features comprise a feature score indicating at least one of an amount, likelihood, or severity of a respective dental feature on a numerical scale;
assigning an oral cavity profile to said person based on a profile framework comprising a plurality of oral cavity profiles, each oral cavity profile being associated with at least one dental feature, respective feature scores of dental features, and optional anamnestic information; and
determining at least one oral care means (6) based on the assigned oral cavity profile (5), said oral care means (6) being one of:
an oral care ingredient (7) statistically associated with an effect on said identified dental features (3) present in the oral cavity (31),
an ingredient combination (8) comprising a plurality of oral care ingredients (7) according to specific proportions or amounts, said ingredient combination (8) being statistically associated with an effect on said identified dental features (3) present in the oral cavity (31), or
an oral care product (9) with at least one mechanical (64), biological (65), or chemical (66) characteristic statistically associated with an effect on said identified dental features (3) present in the oral cavity (31).

2-3. (canceled)

4. A method according to claim 1, wherein determining said at least one oral care means (6) comprises:
performing a severity threshold check for a set of priority dental features (3A) according to a conditional sequence (60) based on the effect of the respective priority dental features (3A) on the oral health of said person (30), wherein each step (61) of said conditional sequence (60) comprises comparing the identified feature score (4) of a respective priority dental feature (3A) for a person (30) to a predefined severity threshold value (22); wherein if the condition is fulfilled an oral care means (6) defined in the respective step is selected for said person (30).

5. A method according to claim 1, wherein determining said at least one oral care means (6) based on the assigned oral cavity profile (5) comprises:
using a decision-tree logic (23) based on dental features (3) and optionally respective feature scores (4) associated with said oral cavity (31) profile.

6. A method according to claim 1, wherein determining said at least one oral care means (6) based on the assigned oral cavity profile (5) comprises:
analyzing dental features (3) and respective feature scores (4) associated with said oral cavity profile (5) using regression analysis (24) where persons (30) are mapped into sub-populations (32) in a statistical space of predefined dimensions, wherein persons (30) with similar oral cavity profiles (5) are part of a same sub-population (32), and likely to benefit from oral care means (6) that have been statistically effective for said sub-population (32).

7. A method according to claim 1, wherein determining said at least one oral care means (6) based on the assigned oral cavity profile (5) comprises:
analyzing said oral cavity profile (5) using a machine learning algorithm (25) trained to identify oral care means (6) likely to be effective based on associated information with said oral cavity profile (5), said associated information comprising dental features (3) and respective feature scores (4), non-image data (2), and optionally follow-up user input.

8. A method according to claim 1, wherein determining the at least one oral care means (6) for an oral cavity profile (5) is based on a treatment framework (26) comprising a plurality of oral care means (6), each oral care means (6) being associated with at least one oral cavity profile (5), based on effectiveness of said oral care means (6) as possible treatment in respect of dental features (3) associated with respective oral cavity profiles (5).

9. A method according to claim 1, wherein the method further comprises presenting said at least one oral care means (6) on a display (48) of said mobile device (40), along with a first match score (33) based on the effectiveness of said at least one oral care means (6) as possible treatment in respect of dental features (3) associated with said oral cavity (31) profile.

10. A method according to claim 9, wherein the method further comprises:
obtaining information related to oral care means (6) currently applied by the person (30);
determining a second match score (34) based on the effectiveness of the currently applied oral care means (6) as possible treatment in respect of dental features (3) associated with the associated oral cavity profile (5) of the person (30); and
presenting said second match score (34) on the display (48) of said mobile device (40).

11. A method according to claim 1, wherein the method further comprises:
presenting said at least one oral care means (6) on a display (48) of said mobile device (40) along with a choice option (35) for the person (30) to “accept” or “reject” the presented oral care means (6); and
if the person (30) chooses to “reject” the presented oral care means (6), repeating the steps of the method to obtain newly determined oral care means (6A), starting either from obtaining the at least one digital image (1), obtaining the non-image data (2), processing the digital image (1), assigning the oral cavity profile (5), or determining the least one oral care means (6); with a rule applied that the previously determined and rejected oral care means (6) are excluded from the options to select from as newly determined oral care means (6A).

12. A computer program product, encoded on a computer-readable storage medium, operable to cause a processor to perform operations according to the method of claim 1.

13. A method of providing a customized oral care product (11) for a person (30), said method comprising:
obtaining at least one digital image (1) of an oral cavity (31) of a person (30) and non-image data (2) comprising anamnestic information associated with said person (30);
determining at least one oral care means (6) for said person (30) based on said at least one digital image (1) and said non-image data (2), by assigning an oral cavity profile (5) to said person (30) according to the method of claim 1; and
manufacturing a customized oral care product (11) for said person (30), said customized oral care product (11) comprising said oral care ingredient (7) or said ingredient combination (8), or corresponding to said oral care product (9) determined as said at least one oral care means (6) according to the method of claim 1.

14. A method according to claim 13, wherein providing said customized oral care product (11) for a person (30) comprises:
determining a plurality of oral cavity profiles (5);
selecting a limited set of common oral cavity profiles (12) from said plurality of determined oral cavity profiles (5);
manufacturing a plurality of Stock Keeping Units, SKUs (13), each SKU (13) comprising a set of oral care products (9) manufactured for a respective common oral cavity profile (12);
matching the assigned oral cavity profile (5) of said person (30) with a closest common oral cavity profile (12); and
providing an oral care product (9) for said person (30) from the SKU (13) manufactured for said closest common oral cavity profile (12).

15. A method according to claim 13, the method further comprising:
obtaining feedback (37) from said person (30) on the effectiveness of the provided customized oral care product (11) on identified dental features (3) present in the oral cavity (31) of said person (30);
determining at least one adjusted oral care means (14) for said person (30), based on both the assigned oral cavity profile (5) of said person (30) and said feedback (37); and
manufacturing an adjusted oral care product (15) for said person (30) comprising or corresponding to said at least one adjusted oral care means (14).

16. A method according to claim 15, wherein obtaining said feedback (37) comprises obtaining follow-up non-image data (16) comprising at least one of follow-up anamnestic information associated with said person (30) covering a period of use of said customized oral care product (11), or perceptive information regarding the perceived effectiveness of the customized oral care product (11) after a period of use; and wherein
determining said at least one adjusted oral care means (14) comprises at least one of comparing the originally obtained anamnestic information with said follow-up anamnestic information, or analyzing said perceptive information.

17. A method according to claim 15, wherein obtaining said feedback (37) comprises:
obtaining at least one follow-up digital image (17) of the oral cavity (31) of said person (30); and
processing said at least one follow-up digital image (17) using a statistical algorithm (20) trained to identify dental features (3) present in an oral cavity (31) based on digital images of a plurality of oral cavities (31), to identify at least one follow-up dental feature (18);
and wherein determining said at least one adjusted oral care means (14) comprises comparing the originally identified dental features (3) present in the oral cavity (31) of said person (30) to said identified follow-up dental features (18).

18. A method according to claim 15, wherein obtaining said feedback (37) comprises obtaining a plurality of feedback (37) from a plurality of persons (30), and selecting relevant feedback (37) for a user based on statistical similarities between said user and said plurality of persons (30).
